(19) United States
(12) Patent Application Publication
(10) Pub. No.: US 2025/0268648 A1
(43) Pub. Date: Aug. 28, 2025

(54) CONTROL METHOD FOR ELECTRICAL TREATMENT DEVICE CAPABLE OF INJECTION AND ELECTRICAL TREATMENT DEVICE

(71) Applicant: SHENZHEN PENINSULA MEDICAL GROUP, Shenzhen (CN)

(72) Inventors: Li LIAO, Shenzhen (CN); Yanan LI, Shenzhen (CN); Xiaobing LEI, Shenzhen (CN); Yi DING, Shenzhen (CN)

(73) Assignee: SHENZHEN PENINSULA MEDICAL GROUP, Shenzhen (CN)

(21) Appl. No.: 19/204,709

(22) Filed: May 12, 2025

Related U.S. Application Data
(63) Continuation of application No. PCT/CN2024/118870, filed on Sep. 13, 2024.

(30) Foreign Application Priority Data
Dec. 28, 2023 (CN) 202311851026.2

(51) Int. Cl. A61B 18/14; A61B 18/00

(52) U.S. Cl. CPC A61B 18/1477; A61B 2018/00404; A61B 2018/00589; A61B 2018/00702; A61B 2018/00875; A61B 2218/002; A61B 2218/007

(57) ABSTRACT

The present application relates to the technical field of device control, and discloses a control method for an electrical treatment device with an injection function and an electrical treatment device. The control method includes: pushing a plurality of the microneedles to move and penetrate a treatment surface of skin by a drive unit; detecting whether the plurality of the microneedles are pushed against a blood vessel by a detection unit; and controlling the plurality of the microneedles to release electric energy and/or perform the drug injection in response to that the plurality of the microneedles are pushed against a blood vessel is detected.

### CROSS-REFERENCE TO RELATED APPLICATIONS

The present application is a continuation application of International Application No. PCT/CN2024/118870, filed on Sep. 13, 2024, which claims priority to Chinese Patent Application No. 202311851026.2, filed on Dec. 28, 2023. The disclosures of the above-mentioned applications are incorporated herein by reference in their entireties.

### TECHNICAL FIELD

The present application relates to the technical field of device control, and in particular to a control method for an electrical treatment device capable of an injection and an electrical treatment device.

### BACKGROUND

With the rapid development of the medical beauty industry, the skin treatment using radiofrequency energy and drug injection treatment are become the choice for an increasing number of people. The radiofrequency treatment is to insert microneedles into the skin to a certain depth, and generate electrical energy by the microneedles, thereby achieving the effect of stimulating the production of collagen and/or ablating fat. The drug injection treatment is to insert the microneedles into the skin tissue to a certain depth, and inject drugs (the drug can be a medicine, a moisturizer, a lotion, or any combination thereof) into the skin by the microneedles to achieve the purpose of cosmetic treatment.

The safety assessment of existing radiofrequency treatment and drug injection treatment is usually carried out by the experience of the operator, which has low safety and affects the treatment effect at the same time.

The above content is only used to assist in understanding the technical solution of the present application and does not mean that the above content is recognized as prior art.

### SUMMARY

The main purpose of the present application is to provide a control method for an electrical treatment device with injection function and an electrical treatment device, aiming to solve the technical problem that the safety assessment in injection treatment in the prior art is usually based on the experience of the operator, which is low safety and affects the treatment effect.

To achieve the above purpose, the present application provides a control method for an electrical treatment device capable of a drug injection, applied to a control device of the electrical treatment device, and the method includes:
pushing a plurality of the microneedles to move and penetrate a treatment surface of skin by a drive unit;
detecting whether the plurality of the microneedles are pushed against a blood vessel by a detection unit; and
controlling the plurality of the microneedles to release electric energy and/or perform the drug injection in response to that the plurality of the microneedles are pushed against a blood vessel is detected.

In an embodiment, the control method for the electrical treatment device further includes:
controlling the plurality of the microneedles to release the electric energy and perform the drug injection in response to that the microneedles are not pushed against the blood vessel is detected.

In an embodiment, said controlling the plurality of the microneedles to release the electric energy and perform the drug injection in response to that the plurality of the microneedles are pushed against the blood vessel is detected includes:
controlling the plurality of the microneedles to release the electric energy and stop the drug injection in response to that the microneedles are pushed against the blood vessel is detected.

In an embodiment, said controlling the plurality of the microneedles to release the electric energy and stop the drug injection in response to that the plurality of the microneedles are pushed against the blood vessel is detected includes:
determining one or more target microneedles in response to that the plurality of microneedles are pushed against the blood vessel is detected; the target microneedles are pushed against the blood vessel; and
controlling the target microneedles to release the electric energy and stop the drug injection, and controlling remaining microneedles to release the electric energy and perform the drug injection; the remaining microneedles are not pushed against the blood vessel.

In an embodiment, before said controlling the target microneedles to release the electric energy and stop the drug injection, and controlling the remaining microneedles to release the electric energy and perform the drug injection, the method further includes:
controlling the target microneedles to release radiofrequency energy for electrocoagulation; and
controlling the target microneedles and the remaining microneedles to release the electric energy and perform the drug injection when said releasing radiofrequency energy for electrocoagulation is completed.

In an embodiment, said controlling the plurality of the microneedles to release the electric energy and stop the drug injection in response to that the plurality of the microneedles are pushed against the blood vessel is detected includes:
controlling the plurality of the microneedles to release radiofrequency energy for electrocoagulation in response to that the microneedles are pushed against the blood vessel is detected; and
controlling the plurality of the microneedles to release the electric energy and perform the drug injection when said releasing radiofrequency energy for electrocoagulation is completed.

In an embodiment, said detecting whether the plurality of the microneedles are pushed against the blood vessel by the detection unit includes:
performing tissue impedance detection among the microneedles in real time by the detection unit, and obtaining a tissue impedance detection value corresponding to the microneedles; and
determining whether the microneedles are pushed against the blood vessel based on the tissue impedance detection value.

In an embodiment, said determining whether the microneedles are pushed against the blood vessel based on the tissue impedance detection value includes:
comparing the tissue impedance detection value with a preset tissue impedance detection threshold;
determining that the microneedles are not pushed against the blood vessel in response to that the tissue impedance detection value does not exceed the preset tissue impedance detection threshold; or
determining that the microneedles are pushed against the blood vessel in response to that the tissue impedance detection value exceeds the preset tissue impedance detection threshold.

In an embodiment, the drive unit is connected to the needle seat and the processor respectively, the detection unit is connected to the processor, the processor is configured to control the needle seat to move forward/backward by the drive unit, and the detection unit comprises an optical detector; said detecting whether the plurality of the microneedles are pushed against the blood vessel by the detection unit includes:
controlling the plurality of the microneedles with aspirated tissue fluid to be withdrawn by the drive unit;
in response to completing the withdrawn, measuring hemoglobin concentration in the aspirated tissue fluid by the optical detector; and
determining whether the microneedles are pushed against the blood vessel based on the hemoglobin concentration.

In addition, to achieve the above purpose, the present application further proposes a control method for an electrical treatment device, applied to a control device of the electrical treatment device, wherein the electrical treatment device comprises a plurality of microneedles, a needle seat, a drive unit, a detection unit, a memory, a processor, and a control program for controlling the electrical treatment device stored in the memory and executable on the processor; one ends of the plurality of the microneedles are provided at the needle seat, the microneedles are connected to an electric energy output assembly, and a hollow channel of each of the microneedles is connected to a liquid storage unit; the drive unit is connected to the needle seat and the processor respectively, the detection unit is connected to the processor, and the processor is configured to control the needle seat to move by the drive unit; the control program for controlling the electrical treatment device is configured to implement the following steps:
pushing a plurality of the microneedles to move and penetrate a treatment surface of skin by a drive unit;
detecting whether the plurality of the microneedles are pushed against a blood vessel by a detection unit; and
controlling the plurality of the microneedles to release electric energy and/or perform the drug injection in response to that the plurality of the microneedles are pushed against a blood vessel is detected.

In addition, to achieve the above purpose, the present application further proposes an electrical treatment device, the electrical treatment device includes a plurality of microneedles, a needle seat, a drive unit, a detection unit, a memory, a processor, and a control program for controlling the electrical treatment device stored in the memory and executable on the processor; one ends of the plurality of the microneedles are provided at the needle seat, the microneedles are connected to an electric energy output assembly, and a hollow channel of each of the microneedles is connected to a liquid storage unit; the drive unit is connected to the needle seat and the processor respectively, the detection unit is connected to the processor, and the processor is configured to control the needle seat to move by the drive unit;
the processor is configured for pushing a plurality of the microneedles to move and penetrate a treatment surface of skin by a drive unit;
the processor is configured for detecting whether the plurality of the microneedles are pushed against a blood vessel by a detection unit; and
the processor is further configured for controlling the plurality of the microneedles to release the electric energy and/or perform the drug injection in response to that the plurality of the microneedles are pushed against the blood vessel is detected.

In the present application, it is disclosed that pushing a plurality of the microneedles to move and penetrate a treatment surface of skin by a drive unit, and the microneedles are used to release the electric energy and/or perform drug injection. During the penetration process, detecting whether the plurality of the microneedles are pushed against a blood vessel by a detection unit; and controlling the plurality of the microneedles to release the electric energy and perform the drug injection in response to that the plurality of the microneedles are pushed against the blood vessel is detected. Compared with the prior art, when performing radiofrequency treatment and drug injection treatment, the microneedles are usually withdrawn after penetrating the skin based on the operator's experience to confirm whether the drug injection position is pushed against the blood vessel, which has low safety; since the present application can detect whether the microneedles are pushed against the blood vessel by the detection unit during the process of the microneedles penetrating the treatment surface, and perform radiofrequency treatment and drug injection treatment based on the detection result, therefore, it can avoid that the radiofrequency treatment and drug injection treatment are still performed when the microneedles are pushed against the blood vessel, thereby improving the safety of treatment.

### BRIEF DESCRIPTION OF THE DRAWINGS

FIG. 1 is a structural schematic view of a control device of an electrical treatment device in a hardware operating environment according to an embodiment of the present application.

FIG. 2 is a flow chart of a control method for an electrical treatment device according to a first embodiment of the present application.

FIG. 3 is a structural schematic view of an electrical treatment device in the control method for the electrical treatment device according to the present application.

FIG. 4 is a flow chart of the control method for the electrical treatment device according to a second embodiment of the present application.

FIG. 5 is a flow chart of a control method for an electrical treatment device according to a third embodiment of the present application.

FIG. 6 is a flow chart of a control method for an electrical treatment device according to an embodiment of the present application.

FIG. 7 is a flow chart of a control method for an electrical treatment device according to an embodiment of the present application.

The realization of the purpose, functional features and advantages of the present application will be further explained in conjunction with the embodiments and with reference to the accompanying drawings.

### DETAILED DESCRIPTION OF THE EMBODIMENTS

The specific embodiments described herein are only used to explain the present application and are not used to limit the present application.

The technical solutions in the embodiments according to the present application will be clearly and completely described below in conjunction with the accompanying drawings in the embodiments according to the present application. Obviously, the described embodiments are only a part of the embodiments according to the present application, and not all of the embodiments. Based on the embodiments in the present application, all other embodiments obtained by those skilled in the art without making creative labor fall within the scope of the present application.

In the embodiment of the present application, the descriptions of “first”, “second” or the like are only for descriptive purposes and cannot be understood as indicating or implying the relative importance or implicitly indicating the quantity of the technical features indicated. Therefore, features defined as “first” and “second” may explicitly or implicitly include at least one of these features. In addition, the technical solutions between various embodiments can be combined with each other, but it is based on that those of ordinary skill in the art can realize. When the combination of technical solutions is contradictory or cannot be realized, it should be considered that such combination of technical solutions does not exist and is not within the protection scope claimed by the present application.

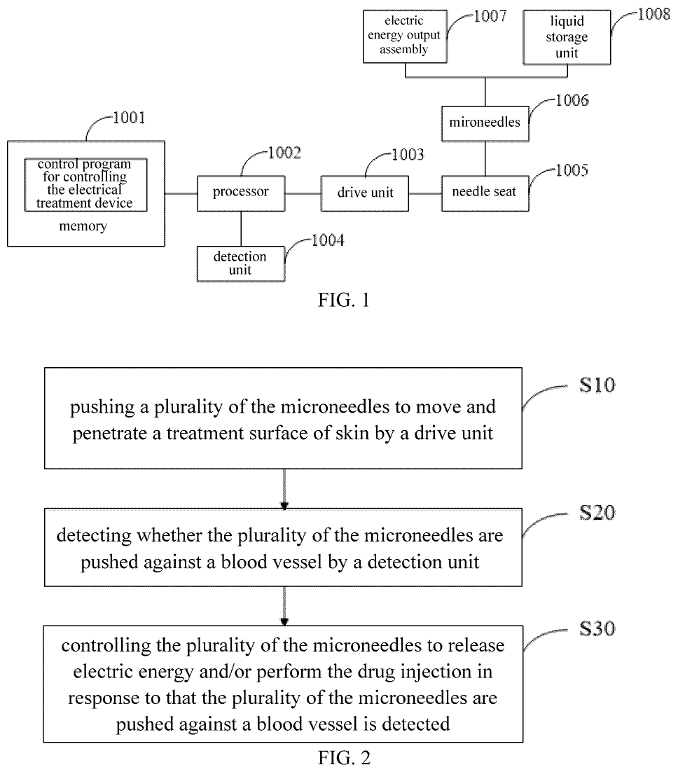

Refer to FIG. 1, FIG. 1 is a structural schematic view of a control device of an electrical treatment device in a hardware operating environment according to an embodiment of the present application.

As shown in FIG. 1, the control device of the electrical treatment device may include a memory 1001, a processor 1002, such as a central processing unit (CPU), a drive unit 1003, a needle seat 1004, and microneedles 1005. The memory 1001 may be a high-speed random access memory (RAM) or a stable non-volatile memory (NVM), such as a disk memory.

The structure shown in FIG. 1 does not constitute a limitation on the control device of the electrical treatment device, and may include more or less components than shown in the figure, or combine certain components, or arrange components differently.

As shown in FIG. 1, the memory 1001 as a storage medium may include a program for controlling the electrical treatment device.

In the control device of the electrical treatment device shown in FIG. 1, the memory 1001 and the processor 1002 in the control device of the electrical treatment device of the present application can be provided in the control device of the electrical treatment device. The control device of the electrical treatment device calls the program for controlling the electrical treatment device stored in the memory 1001 by the processor 1002, and executes the control method for the electrical treatment device provided in the embodiment of the present application.

The embodiment of the present application provides a control method for an electrical treatment device. Refer to FIG. 2, FIG. 2 is a flow chart of a control method for an electrical treatment device according to the first embodiment of the present application.

In this embodiment, the method is applied to a control device of the electrical treatment device, the device includes a plurality of microneedles 1006, a needle seat 1005, a drive unit 1003, a detection unit 1004, a memory 1001, a processor 1002, and a control program for controlling the electrical treatment device stored in the memory 1001 and executable on the processor 1002; one ends of the plurality of the microneedles are provided at the needle seat 1005, the microneedles 1006 are connected to an electric energy output assembly 1007, a hollow channel of each of the microneedles is connected to a liquid storage unit 1008, and the control program for controlling the electrical treatment device is configured to implement the following steps.

Step S10: pushing a plurality of the microneedles to move and penetrate a treatment surface of skin by a drive unit.

The execution subject of the method of this embodiment may be a control device that controls the electrical treatment device to perform radiofrequency treatment and drug injection treatment on the user, or a control system of other electrical treatment devices that can achieve the same or similar functions and include the control device. Here, the control device of the electrical treatment device (hereinafter referred to as the device) is used to specifically describe the control method for the electrical treatment device provided in this embodiment and the following embodiments.

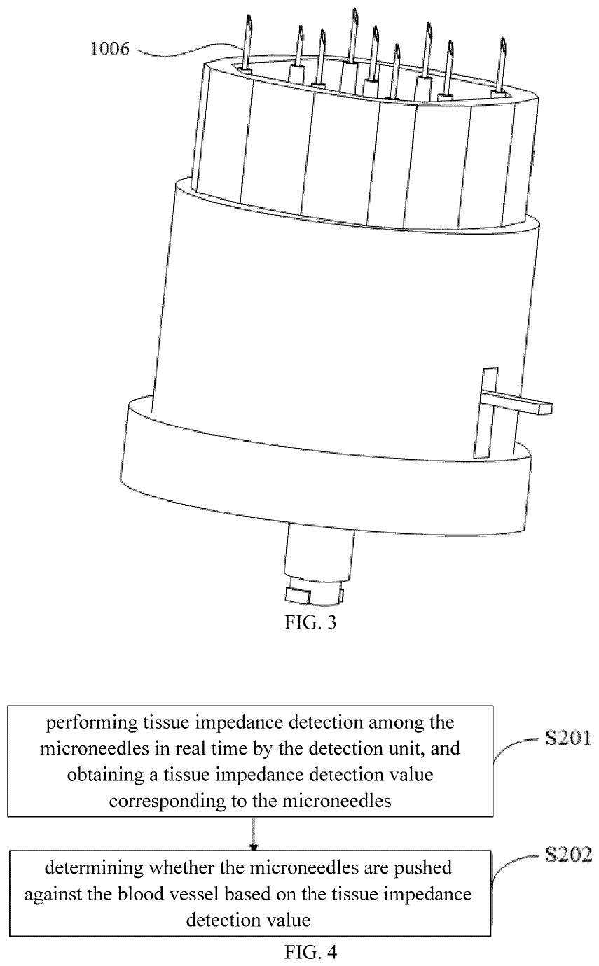

Refer to FIG. 3, FIG. 3 is a structural schematic view of an electrical treatment device in the control method for the electrical treatment device according to the present application. As shown in FIG. 3, the present application can achieve radiofrequency treatment and drug injection treatment on the user by controlling the plurality of the microneedles 1006 in the electrical treatment device to release electrical energy and perform drug injection.

The above-mentioned drive unit can be a structure for driving the needle seat and then controlling the plurality of the microneedles provided on the needle seat. In this embodiment, the drive unit can include but is not limited to a motor, that is, in this embodiment, the microneedles can be controlled by the motor to penetrate the skin of the treatment object. The treatment surface can be any object that undergoes radiofrequency treatment and drug injection treatment.

The microneedles in this embodiment can connect to an electric energy output assembly (such as a radiofrequency power supply), so that electrical energy can be generated after penetrating the skin of the treatment surface, thereby ablating fat, spots, stimulating the production of collagen, and achieving the purpose of radiofrequency treatment. At the same time, since the interior of each hollow microneedle is provided with a hollow channel, the plurality of hollow channels of the microneedles can connect to the liquid storage unit, so that after penetrating the skin of the treatment object, the injectant stored in the liquid storage unit can be controlled to be injected into the skin of the treatment object via the hollow channel, thereby achieving the purpose of injection treatment. The injectant includes solutions, compound solutions, suspensions, gels, etc.; specific ingredients include non-cross-linked nutrients such as hyaluronic acid, collagen, etc. that improve skin condition.

In this embodiment, the drive unit (such as a motor) can be used to control the microneedles to release the injectant to perform drug injection treatment on the treatment surface. The drive unit for controlling the plurality of the microneedles to penetrate and the drive unit for controlling the plurality of the microneedles to release the injectant are different drive units.

The two ends of the microneedles can be a connection end and a treatment end respectively. The connection ends of the microneedles are connected to the needle seat, and the treatment end can be provided to be suspended. When treating the treatment surface, the needle seat can be controlled by the motor to control the treatment ends of the microneedles to penetrate the skin of the treatment surface.

Step S20: detecting whether the plurality of the microneedles are pushed against a blood vessel by a detection unit.

It should be noted that the term “push against” refers to that during pushing the microneedles to move underneath the treatment surface (TS), pushing the plurality of the microneedles against the blood vessel while contacting and/or penetrating the vessel with the plurality of microneedles may cause a data changed that is detected by the detection unit.

The detection unit can be a structure for detecting whether the plurality of the microneedles are pushed against the blood vessel of the treatment surface. The detection unit in this embodiment can be used for detecting a real-time impedance of the treatment tissue and detecting light underneath the treatment surface, so that the device can determine whether the microneedles are contacted and/or penetrated into the blood vessel by the tissue impedance detection result or the light detection result.

Step S30: controlling the plurality of the microneedles to release electric energy and/or perform the drug injection in response to that the plurality of the microneedles are pushed against a blood vessel is detected.

When performing radiofrequency treatment and drug injection treatment by microneedles, there may be a situation where the microneedles are pushed against the blood vessel, at this time, if continuing the drug injection, the drug would be potentially injected into the blood vessel, which is less safe and has a poor treatment effect. Therefore, this embodiment can detect whether the microneedles are pushed against the blood vessel in real time during the process of the microneedles penetrating the treatment surface by the detection unit, and treat the treatment surface based on the corresponding detection result, thereby improving the safety of treatment, and ensuring the treatment effect simultaneously.

In this embodiment, controlling the plurality of the microneedles to release the electric energy and perform the drug injection in response to that the microneedles are not pushed against the blood vessel is detected.

If the detection result shows that the microneedles are not pushed against the blood vessel, it can be that controlling the plurality of the microneedles to release electric energy and perform drug injection, so as to perform radiofrequency treatment and drug injection treatment on the treatment surface.

In this embodiment, controlling the plurality of the microneedles to release the electric energy and stop the drug injection in response to that the microneedles are pushed against the blood vessel is detected.

If the detection result shows that the microneedles are pushed against the blood vessel, at this time, to prevent the drug would be potentially injected into the vessel, controlling the plurality of the microneedles to stop releasing electric energy and injecting, thus preventing the injectant from being injected into the blood vessel, and ensuring medical safety.

Furthermore, refer to FIG. 6, in order to achieve precise treatment of the microneedles, the step S30 includes:

Step S301: determining one or more target microneedles in response to that the plurality of microneedles are pushed against the blood vessel is detected; the target microneedles are pushed against the blood vessel.

The above-mentioned target microneedles may be any one microneedle on the needle seat that is pushed against the blood vessel. The microneedles in this embodiment may be arranged in an array, so that the detection unit may accurately detect whether each microneedle is pushed against the blood vessel on the treatment surface. If every microneedle is detected that it is pushed against the blood vessel, all microneedles that are pushed against the blood vessel may be determined as the target microneedles.

Step S302: controlling the target microneedles to release the electric energy and stop the drug injection, and controlling remaining microneedles to release the electric energy and perform the drug injection; the remaining microneedles are not pushed against the blood vessel.

The above-mentioned remaining microneedles may be any one microneedle on the needle seat that is not pushed against the blood vessel. It should be understood, the device can determine all microneedles that are pushed against the blood vessel as target microneedles according to the detection results, and determine all microneedles that are not pushed against the blood vessel as remaining microneedles.

In this embodiment, in order to achieve radiofrequency treatment and drug injection treatment while ensuring medical safety, this embodiment can stop the radiofrequency release and drug injection of all target microneedles that are pushed against the blood vessel under the treatment surface, and control all remaining microneedles that are not pushed against the blood vessel to release electric energy and perform drug injection, thereby achieving precise treatment of microneedles.

Furthermore, in order to continue treatment after the microneedles are pushed against the blood vessel, before step S302, the method further includes: controlling the target microneedles to release radiofrequency energy for electrocoagulation; and controlling the target microneedles and the remaining microneedles to release the electric energy and perform the drug injection when said releasing radiofrequency energy for electrocoagulation is completed.

The above-mentioned radiofrequency energy can be used for electrocoagulation, that is, performing electrotherapy on the blood vessel in the subcutaneous tissue to coagulate the blood vessel, so as to prevent the drug injection solution from entering the blood via the potential ruptured blood vessel, or prevent the blood flowing outside from the blood vessel to be coagulated. The radiofrequency energy can be radiofrequency energy of relatively high frequency. In this embodiment, the purpose of electrocoagulation of the blood vessel can be achieved by releasing radiofrequency energy by the microneedles.

In this embodiment, if microneedles are pushed against the blood vessel, it can be that controlling the target microneedles that are pushed against the blood vessel to release radiofrequency energy to coagulate the blood vessel, and when the blood vessel coagulates, controlling all microneedles (including the target microneedles and the remaining microneedles) to release electric energy (such as, the radiofrequency energy is used to perform radiofrequency treatment on the treatment surface) and perform drug injection, so as to perform radiofrequency treatment and drug injection treatment on the treatment surface.

Furthermore, in order to improve the treatment efficiency, the step S30 further includes: controlling the plurality of the microneedles to release radiofrequency energy for electrocoagulation in response to that the microneedles are pushed against the blood vessel is detected; and controlling the plurality of the microneedles to release the electric energy and perform the drug injection when said releasing radiofrequency energy for electrocoagulation is completed.

In this embodiment, if microneedles are pushed against the blood vessel, in order to improve the treatment efficiency, it is possible not to detect which one or more microneedles are pushed against the blood vessel, and directly controlling all microneedles to release radiofrequency energy to electrocoagulate the blood vessel, that is controlling all microneedles to release electric energy for coagulating the blood vessel and thus perform drug injection when the blood vessel is coagulated, further performing radiofrequency treatment and drug injection treatment on the treatment surface with predetermined parameters.

The present embodiments disclose that pushing a plurality of the microneedles to move and penetrate a treatment surface of skin by a drive unit, and the microneedles are used to release electric energy and/or perform drug injection. During the penetration process, detecting whether the plurality of the microneedles are pushed against a blood vessel by a detection unit; and controlling the plurality of the microneedles to release electric energy and perform the drug injection in response to that the plurality of the microneedles are pushed against the blood vessel is detected. Compared with the prior art, when performing radiofrequency treatment and drug injection treatment, the microneedles are usually withdrawn after penetrating the skin based on the operator's experience to confirm whether one or more microneedles are pushed against the blood vessel, which brings sort of uncertainty and low safety during treatment; since the present application can detect whether the microneedles are pushed against the blood vessel by the detection unit during the process of the microneedles penetrating the treatment surface, and perform radiofrequency treatment and drug injection treatment based on the detection result, therefore, it can avoid that the radiofrequency treatment and drug injection treatment are still performed when the microneedles are pushed against the blood vessel, thereby improving the safety of treatment. Meanwhile, when the microneedles are pushed against the blood vessel, the present embodiment determines the target microneedles that are pushed against the blood vessel according to the detection result, controls the target microneedles to stop releasing electric energy and injecting, and controls the remaining microneedles to release electric energy and/or inject, thereby achieving radiofrequency treatment and drug injection treatment while ensuring medical safety.

Refer to FIG. 4, FIG. 4 is a flow chart of the control method for the electrical treatment device according to a second embodiment of the present application.

Based on the above-mentioned first embodiment, in order to accurately determine whether the microneedles are pushed against the blood vessel and improve the safety of treatment, in this embodiment, the step S20 includes:

Step S201: performing tissue impedance detection among the microneedles in real time by the detection unit, and obtaining a tissue impedance detection value corresponding to the microneedles.

The above-mentioned tissue electrical impedance may indicate the changes of the tissue environment under the skin among the microneedles detected by the detection unit.

Step S202: determining whether the microneedles are pushed against the blood vessel based on the tissue impedance detection value.

Since the impedance values among the microneedles in different layers of the skin and in the blood are different, in this embodiment, during the process of the microneedles penetrating the treatment surface, it can be that detecting the impedance value among the microneedles in real time by the detection unit, and determining whether the microneedles are pushed against the blood vessel according to the impedance value.

Furthermore, the step S202 includes: comparing the tissue impedance detection value with a preset tissue impedance detection threshold; determining that the microneedles are not pushed against the blood vessel in response to that the tissue impedance detection value does not exceed the preset tissue impedance detection threshold; or determining that the microneedles are pushed against the blood vessel in response to that the tissue impedance detection value exceeds the preset tissue impedance detection threshold.

The above-mentioned preset tissue impedance detection threshold may be an impedance maximum/minimum value related to the human skin tissue that is preset and used to indicate whether the microneedles are pushed against a blood vessel since the impedance of the blood vessel is different from the impedance of the skin tissue. In this embodiment, if the current tissue impedance detection value of the microneedles does not exceed the preset tissue impedance detection threshold, it indicates that the microneedles are not pushed against the blood vessel; if the current tissue impedance detection value of the microneedles exceeds the preset tissue impedance detection threshold, it indicates that the microneedles are pushed against the blood vessel. In addition, the present embodiment can further pre-set an tissue impedance detection value range, and determine whether the tissue impedance detection value is within the tissue impedance detection value range; if the tissue impedance detection value is within the tissue impedance detection value range, it indicates that the microneedles currently are pushed against the blood vessel; if the tissue impedance detection value is not within the tissue impedance detection value range, it indicates that the microneedles currently are not pushed against the blood vessel, thereby accurately determining whether the microneedles are pushed against the blood vessel, thus improving treatment safety.

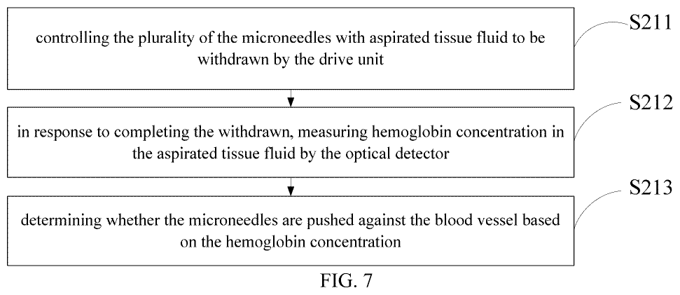

Furthermore, this embodiment can further determine whether the microneedles are pushed against a blood vessel by light detection. If adopting the light detection to perform detection, the drive unit 1003 is connected to the needle seat 1005 and the processor 1002 respectively, the detection unit 1005 is connected to the processor 1002, the processor 1002 is configured to control the needle seat 1005 to move by the drive unit 1003, and the detection unit includes a optical detector; refer to FIG. 7, the step S20 further includes:

Step S211: controlling the plurality of the microneedles with aspirated tissue fluid to be withdrawn by the drive unit.

Step S212: in response to completing the withdrawn, measuring hemoglobin concentration in the aspirated tissue fluid by the optical detector.

In this embodiment, an optical detector is used to measure hemoglobin concentration in tissue fluid aspirated by the microneedles when they are withdrawn from the tissue. In other words, the optical detector can detect the hemoglobin concentration in the tissue fluid by using visible or near-infrared light to realize bleeding monitoring in minimally invasive surgery fluid analysis. The reason is that oxyhemoglobin and deoxyhemoglobin exhibit characteristic absorption peaks in the visible (500 nm to 700 nm) and near-infrared (700 nm to 1000 nm) ranges, the optical path design may use the reflection mode, that means, light is scattered and reflected back from the tissue aspirated fluid to measure the hemoglobin concentration, therefore, determining whether there is blood in the aspirated tissue fluid.

Step S213: determining whether the microneedles are pushed against the blood vessel based on the hemoglobin concentration.

If there is blood in the tissue fluid aspirated by the microneedles, it indicates that the microneedles have penetrated the blood vessel under the skin; if there is no blood in the tissue fluid aspirated by the microneedles, it indicates that the microneedles are not pushed against the blood vessel currently.

This embodiment performs tissue impedance detection among the microneedles in real time by the detection unit, obtains the tissue impedance detection value corresponding to the microneedles, and determines whether the microneedles are pushed against the blood vessel based on the tissue impedance detection value, thereby accurately determining whether the microneedles are pushed against the blood vessel, thus improving the safety of treatment. In addition, this embodiment further can control the microneedles to be withdrawn by the drive unit along with the tissue fluid aspirated by the microneedles, detect whether there is blood in the aspirated tissue fluid by the light detection unit, and then determines whether the microneedles are pushed against the blood vessel according to the detection result, thereby improving the flexibility of detection.

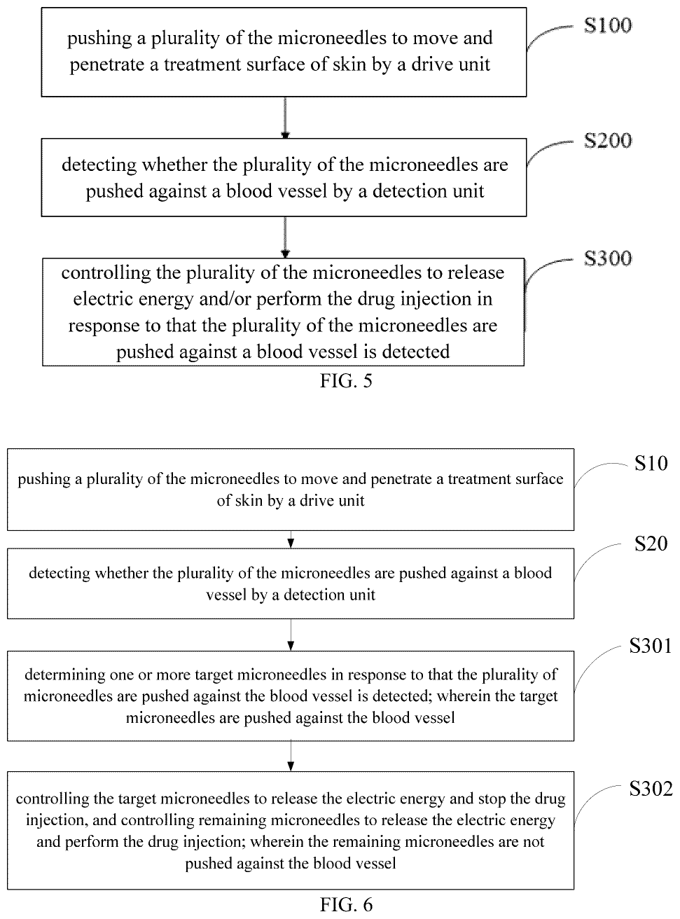

Refer to FIG. 5, FIG. 5 is a flow chart of a control method for an electrical treatment device according to a third embodiment of the present application.

Based on the above-mentioned embodiments, in this embodiment, the method is applied to a control device in the electrical treatment device, the electrical treatment device includes a plurality of microneedles 1006, a needle seat 1005, a drive unit 1003, a detection unit 1004, a memory 1001, a processor 1002, and a control program for controlling the electrical treatment device stored in the memory 1001 and executable on the processor 1002; one ends of the plurality of the microneedles are provided at the needle seat 1005, the microneedles 1006 are connected to an electric energy output assembly 1007, and a hollow channel of each of the microneedles is connected to a liquid storage unit 1008; the drive unit 1003 is connected to the needle seat 1005 and the processor 1002 respectively, the detection unit 1005 is connected to the processor 1002, and the processor 1002 is configured to control the needle seat 1005 to move by the drive unit 1003; the control program for controlling the electrical treatment device is configured to implement the following steps.

Step S100: pushing a plurality of the microneedles to move and penetrate a treatment surface of skin by a drive unit.

Step S200: detecting whether the plurality of the microneedles are pushed against a blood vessel by a detection unit.

Step S300: controlling the plurality of the microneedles to release electric energy and/or perform the drug injection in response to that the plurality of the microneedles are pushed against a blood vessel is detected.

In this embodiment, in order to improve the efficiency of radiofrequency treatment and drug injection treatment while ensuring medical safety, after the microneedles penetrate the treatment surface, it is not detected whether the microneedles are pushed against the blood vessel, but directly controlling the plurality of the microneedles to release radiofrequency energy to coagulate the blood vessel to prevent the drug from being injected into the blood vessel; after completing the electrocoagulation, continuing to control the microneedles to release electric energy and perform drug injection, so as to perform radiofrequency treatment and drug injection treatment on the treatment surface.

This embodiment controls a plurality of the microneedles to penetrate the treatment surface by the drive unit, and controls the microneedles to release radiofrequency energy to coagulate the blood vessel after completing the penetration, so as to control the microneedles to release electric energy and/or perform drug injection after completing the electrocoagulation, so that it can improve the efficiency of radiofrequency treatment and drug injection treatment while ensuring medical safety.

Based on the above-mentioned embodiments of the control method for an electrical treatment device, a first embodiment of the electrical treatment device of the present application is described hereinafter.

In this embodiment, the electrical treatment device includes a plurality of microneedles 1006, a needle seat 1005, a drive unit 1003, a detection unit 1004, a memory 1001, a processor 1002, and a control program for controlling the electrical treatment device stored in the memory 1001 and executable on the processor 1002; one ends of the plurality of the microneedles are provided at the needle seat 1005, the microneedles 1006 are connected to an electric energy output assembly 1007, and a hollow channel of each of the microneedles is connected to a liquid storage unit 1008; the drive unit 1003 is connected to the needle seat 1005 and the processor 1002 respectively, the detection unit 1004 is connected to the processor 1002, and the processor 1002 is configured to control the needle seat 1005 to move by the drive unit 1003.

The processor 1002 is configured for pushing a plurality of the microneedles 1006 to move and penetrate a treatment surface of skin by a drive unit 1003. The microneedles 1006 are configured for releasing electric energy and/or performing drug injection.

The processor 1002 is configured for detecting whether the plurality of the microneedles 1006 are pushed against a blood vessel by a detection unit 1004.

The processor 1002 is further configured for controlling the plurality of the microneedles 1006 to release electric energy and/or perform the drug injection in response to that the plurality of the microneedles are pushed against the blood vessel is detected.

This embodiment discloses that pushing a plurality of the microneedles to move and penetrate a treatment surface of skin by a drive unit, and the microneedles are configured for releasing electric energy and/or performing drug injection; during the penetration process, detecting whether the plurality of the microneedles are pushed against a blood vessel by a detection unit, controlling the plurality of the microneedles to release electric energy and/or perform the drug injection in response to that the plurality of the microneedles are pushed against the blood vessel is detected. Compared with the prior art, when performing radiofrequency treatment and drug injection treatment, the microneedles are usually withdrawn after penetrating the skin based on the operator's experience to confirm whether the drug injection position is pushed against the blood vessel, which has low safety; since the present application can detect whether the microneedles are pushed against the blood vessel by the detection unit during the process of the microneedles penetrating the treatment surface, and perform radiofrequency treatment and drug injection treatment based on the detection result, therefore, it can avoid that the radiofrequency treatment and drug injection treatment are still performed when the microneedles are pushed against the blood vessel, thereby improving the safety of treatment.

Based on the above-mentioned embodiments of the control method for the electrical treatment device, the second embodiment of the electrical treatment device of the present application is described hereinafter.

In this embodiment, the electrical treatment device includes a plurality of microneedles 1006, a needle seat 1005, a drive unit 1003, a detection unit 1004, a memory 1001, a processor 1002, and a control program for controlling the electrical treatment device stored in the memory 1001 and executable on the processor 1002; one ends of the plurality of the microneedles are provided at the needle seat 1005, the microneedles 1006 are connected to an electric energy output assembly 1007, and a hollow channel of each of the microneedles are connected to a liquid storage unit 1008; the drive unit 1003 is connected to the needle seat 1005 and the processor 1002 respectively, the detection unit 1004 is connected to the processor 1002, and the processor 1002 is configured to control the needle seat 1005 to move by the drive unit 1003.

The processor 1002 is configured for pushing a plurality of the microneedles 1006 to move and penetrate a treatment surface of skin by a drive unit 1003.

The processor 1002 is configured for controlling the plurality of the microneedles 1006 to release radiofrequency energy for electrocoagulation to coagulate a blood vessel.

The processor 1002 is further configured for controlling the plurality of the microneedles 1006 to release electric energy and/or perform drug injection in response to completing the electrocoagulation.

The embodiment controls the drive unit 1003 to push the plurality of the microneedles 1006 to penetrate the treatment surface, controls the microneedles 1006 to release radiofrequency energy to electrocoagulate the blood vessel after completing the penetration of the treatment surface, and controls the microneedles to release electric energy and perform drug injection after completing the electrocoagulation, so that it can ensure medical safety while improving the efficiency of radiofrequency treatment and drug injection treatment.

In the present application, the terms “include”, “comprise” or any other variation thereof are intended to cover a non-exclusive inclusion, such that a process, method, article or system that includes a list of elements not only includes those elements, but also includes other elements not expressly listed, or also includes elements inherent to the process, method, article or system. In a case of without further restrictions, an element defined by the statement “includes . . . ” does not exclude the existence of other identical elements in a process, method, article or system that includes this element.

The serial numbers of the embodiments of the present application are only for description and do not represent the advantages and disadvantages of the embodiments.

By the description of the above implementation mode, those skilled in the art can clearly understand that the above embodiment method can be implemented by means of software plus the necessary general hardware platform mode. Certainly, it can also be implemented by hardware, but in many cases the former is a better implementation mode. Based on this understanding, the technical solution of the present application, or the part that contributes to the prior art, can be embodied in the form of a software product, which is stored in a storage medium (such as a read-only memory/random access memory, a disk, or an optical disk), and includes several instructions for enabling a terminal device (which can be a mobile phone, a computer, a server, or a network device, etc.) to execute the methods of each embodiment of the present application.

The above are only some embodiments of the present application, and do not limit the scope of the present application. Any equivalent structure or equivalent process transformation made using the contents of the specification and drawings of the present application, or directly or indirectly used in other related technical fields, are also included in the scope of the present application.

What is claimed is:

1. A control method for an electrical treatment device capable of a drug injection, applied to a control device of the electrical treatment device, comprising:
pushing a plurality of the microneedles to move and penetrate a treatment surface of skin by a drive unit;
detecting whether the plurality of the microneedles are pushed against a blood vessel by a detection unit; and
controlling the plurality of the microneedles to release electric energy and/or perform the drug injection in response to that the plurality of the microneedles are pushed against a blood vessel is detected.

2. The control method for the electrical treatment device according to claim 1, further comprising:
controlling the plurality of the microneedles to release the electric energy and perform the drug injection in response to that the microneedles are not pushed against the blood vessel is detected.

3. The control method for the electrical treatment device according to claim 1, wherein said controlling the plurality of the microneedles to release the electric energy and perform the drug injection in response to that the plurality of the microneedles are pushed against the blood vessel is detected comprises:
controlling the plurality of the microneedles to release the electric energy and stop the drug injection in response to that the microneedles are pushed against the blood vessel is detected.

4. The control method for the electrical treatment device according to claim 3, wherein said controlling the plurality of the microneedles to release the electric energy and stop the drug injection in response to that the plurality of the microneedles are pushed against the blood vessel is detected comprises:
determining one or more target microneedles in response to that the plurality of microneedles are pushed against the blood vessel is detected; wherein the target microneedles are pushed against the blood vessel; and
controlling the target microneedles to release the electric energy and stop the drug injection, and controlling remaining microneedles to release the electric energy and perform the drug injection; wherein the remaining microneedles are not pushed against the blood vessel.

5. The control method for the electrical treatment device according to claim 4, wherein before said controlling the target microneedles to release the electric energy and stop the drug injection, and controlling the remaining microneedles to release the electric energy and perform the drug injection, the method further comprises:
controlling the target microneedles to release radiofrequency energy for electrocoagulation; and
controlling the target microneedles and the remaining microneedles to release the electric energy and perform the drug injection when said releasing radiofrequency energy for electrocoagulation is completed.

6. The control method for the electrical treatment device according to claim 3, wherein said controlling the plurality of the microneedles to release the electric energy and stop the drug injection in response to that the plurality of the microneedles are pushed against the blood vessel is detected comprises:
controlling the plurality of the microneedles to release radiofrequency energy for electrocoagulation in response to that the microneedles are pushed against the blood vessel is detected; and
controlling the plurality of the microneedles to release the electric energy and perform the drug injection when said releasing radiofrequency energy for electrocoagulation is completed.

7. The control method for the electrical treatment device according to claim 1, wherein said detecting whether the plurality of the microneedles are pushed against the blood vessel by the detection unit comprises:
performing tissue impedance detection among the microneedles in real time by the detection unit, and obtaining a tissue impedance detection value corresponding to the microneedles; and
determining whether the microneedles are pushed against the blood vessel based on the tissue impedance detection value.

8. The control method for the electrical treatment device according to claim 7, wherein said determining whether the microneedles are pushed against the blood vessel based on the tissue impedance detection value comprises:
comparing the tissue impedance detection value with a preset tissue impedance detection threshold;
determining that the microneedles are not pushed against the blood vessel in response to that the tissue impedance detection value does not exceed the preset tissue impedance detection threshold; or
determining that the microneedles are pushed against the blood vessel in response to that the tissue impedance detection value exceeds the preset tissue impedance detection threshold.

9. The control method for the electrical treatment device according to claim 1, wherein the drive unit is connected to the needle seat and the processor respectively, the detection unit is connected to the processor, the processor is configured to control the needle seat to move forward/backward by the drive unit, and the detection unit comprises an optical detector; said detecting whether the plurality of the microneedles are pushed against the blood vessel by the detection unit comprises:
controlling the plurality of the microneedles with aspirated tissue fluid to be withdrawn by the drive unit;
in response to completing the withdrawn, measuring hemoglobin concentration in the aspirated tissue fluid by the optical detector; and
determining whether the microneedles are pushed against the blood vessel based on the hemoglobin concentration.

10. A control method for an electrical treatment device, applied to a control device of the electrical treatment device, wherein the electrical treatment device comprises a plurality of microneedles, a needle seat, a drive unit, a detection unit, a memory, a processor, and a control program for controlling the electrical treatment device stored in the memory and executable on the processor; one ends of the plurality of the microneedles are provided at the needle seat, the microneedles are connected to an electric energy output assembly, and a hollow channel of each of the microneedles is connected to a liquid storage unit; the drive unit is connected to the needle seat and the processor respectively, the detection unit is connected to the processor, and the processor is configured to control the needle seat to move by the drive unit; the control program for controlling the electrical treatment device is configured to implement the following steps:
pushing a plurality of the microneedles to move and penetrate a treatment surface of skin by a drive unit;
detecting whether the plurality of the microneedles are pushed against a blood vessel by a detection unit; and
controlling the plurality of the microneedles to release electric energy and/or perform the drug injection in response to that the plurality of the microneedles are pushed against a blood vessel is detected.

11. An electrical treatment device, wherein the electrical treatment device comprises a plurality of microneedles, a needle seat, a drive unit, a detection unit, a memory, a processor, and a control program for controlling the electrical treatment device stored in the memory and executable on the processor; one ends of the plurality of the microneedles are provided at the needle seat, the microneedles are connected to an electric energy output assembly, and a hollow channel of each of the microneedles is connected to a liquid storage unit; the drive unit is connected to the needle seat and the processor respectively, the detection unit is connected to the processor, and the processor is configured to control the needle seat to move by the drive unit;
wherein the processor is configured for pushing a plurality of the microneedles to move and penetrate a treatment surface of skin by a drive unit;
wherein the processor is configured for detecting whether the plurality of the microneedles are pushed against a blood vessel by a detection unit; and
wherein the processor is further configured for controlling the plurality of the microneedles to release electric energy and/or perform the drug injection in response to that the plurality of the microneedles are pushed against a blood vessel is detected.

12. The electrical treatment device according to claim 11, wherein the processor is further configured for controlling the plurality of the microneedles to release the electric energy and perform the drug injection in response to that the microneedles are not pushed against the blood vessel is detected.

13. The electrical treatment device according to claim 11, wherein the processor is further configured for controlling the plurality of the microneedles to release the electric energy and stop the drug injection in response to that the microneedles are pushed against the blood vessel is detected.

14. The electrical treatment device according to claim 13, wherein the processor is further configured for:
determining one or more target microneedles in response to that the plurality of microneedles are pushed against the blood vessel is detected; wherein the target microneedles are pushed against the blood vessel; and
controlling the target microneedles to release the electric energy and stop the drug injection, and controlling remaining microneedles to release the electric energy and perform the drug injection; wherein the remaining microneedles are not pushed against the blood vessel.

15. The electrical treatment device according to claim 14, wherein the processor is further configured for:
controlling the target microneedles to release radiofrequency energy for electrocoagulation; and
controlling the target microneedles and the remaining microneedles to release the electric energy and perform the drug injection when said releasing radiofrequency energy for electrocoagulation is completed.

16. The electrical treatment device according to claim 13, wherein the processor is further configured for:
controlling the plurality of the microneedles to release radiofrequency energy for electrocoagulation in response to that the microneedles are pushed against the blood vessel is detected; and
controlling the plurality of the microneedles to release the electric energy and perform the drug injection when said releasing radiofrequency energy for electrocoagulation is completed.

17. The electrical treatment device according to claim 11, wherein the processor is further configured for:
performing tissue impedance detection among the microneedles in real time by the detection unit, and obtaining a tissue impedance detection value corresponding to the microneedles; and
determining whether the microneedles are pushed against the blood vessel based on the tissue impedance detection value.

18. The electrical treatment device according to claim 17, wherein the processor is further configured for:
comparing the tissue impedance detection value with a preset tissue impedance detection threshold;
determining that the microneedles are not pushed against the blood vessel in response to that the tissue impedance detection value does not exceed the preset tissue impedance detection threshold; or
determining that the microneedles are pushed against the blood vessel in response to that the tissue impedance detection value exceeds the preset tissue impedance detection threshold.

19. The electrical treatment device according to claim 11, wherein the drive unit is connected to the needle seat and the processor respectively, the detection unit is connected to the processor, the processor is configured to control the needle seat to move forward/backward by the drive unit, and the detection unit comprises a optical detector; wherein the processor is further configured for:
controlling the plurality of the microneedles with aspirated tissue fluid to be withdrawn by the drive unit;
in response to completing the withdrawn, measuring hemoglobin concentration in the aspirated tissue fluid by the optical detector; and
determining whether the microneedles are pushed against the blood vessel based on the hemoglobin concentration.
